(19) United States
(12) Patent Application Publication
(10) Pub. No.: US 2022/0305481 A1
(43) Pub. Date: Sep. 29, 2022

(54) METHOD FOR PRODUCING CATALYST FOR PRODUCTION OF METHACRYLIC ACID, METHOD FOR PRODUCING METHACRYLIC ACID, METHOD FOR PRODUCING METHACRYLIC ACID ESTER, AND APPARATUS FOR PRODUCING CATALYST FOR PRODUCTION OF METHACRYLIC ACID

(71) Applicant: Mitsubishi Chemical Corporation, Tokyo (JP)

(72) Inventors: Yuki Kato, Tokyo (JP); Masahide Kondo, Tokyo (JP)

(73) Assignee: Mitsubishi Chemical Corporation, Tokyo (JP)

(21) Appl. No.: 17/831,711

(22) Filed: Jun. 3, 2022

Related U.S. Application Data
(63) Continuation of application No. PCT/JP2021/000544, filed on Jan. 8, 2021.

(30) Foreign Application Priority Data
Jan. 8, 2020 (JP) 2020-001485

(51) Int. Cl. B01J 37/03; B01J 37/04; B01J 35/02; B01J 27/199; C07C 51/25; C07C 67/08

(52) U.S. Cl. CPC B01J 37/038; B01J 37/04; B01J 35/023; B01J 27/199; C07C 51/252; C07C 67/08

(57) ABSTRACT

The present invention provides a production method which is capable of stably producing a catalyst that enables a production of methacrylic acid with high selectivity. A method of producing a catalyst used for a production of methacrylic acid includes (i) preparing a slurry A1 containing a heteropolyacid containing at least phosphorus and molybdenum or a salt of the heteropolyacid containing at least phosphorus and molybdenum, (ii) preparing a slurry A2 satisfying the following Formula (I) and Formula (II) using the slurry A1, (iii) mixing the slurry A2 and a raw material liquid B containing a cationic raw material to prepare a slurry C, and (iv) drying the slurry C, α_{A2}/α_{A1}≤0.95 (I), 2≤D_{A2}≤50 (II), wherein, in Formula (I), α_{A1} represents a half-value width (μm) of a particle size distribution of the slurry A1, α_{A2} represents a half-value width (μm) of a particle size distribution of the slurry A2, and in Formula (II), D_{A2} represents a median diameter (μm) of the particle size distribution of the slurry A2.

### TECHNICAL FIELD

The present invention relates to a method of producing a catalyst used for a production of methacrylic acid, a method of producing methacrylic acid, a method of producing a methacrylic acid ester, and an apparatus for producing a catalyst used for a production of methacrylic acid.

### BACKGROUND ART

As a catalyst for producing methacrylic acid for producing methacrylic acid by oxidizing methacrolein, (hereinafter, also simply referred to as “catalyst”) a heteropolyacid-based catalyst containing molybdenum and phosphorus is known. Such a heteropolyacid-based catalyst includes a proton-type heteropolyacid in which counter cations are protons, and a heteropolyacid salt in which a part of the protons is replaced with a cation other than the protons (hereinafter, a proton-type heteropolyacid is also simply referred to as “heteropolyacid”, a proton-type heteropolyacid and/or a heteropolyacid salt is also referred to as “heteropolyacid (salt)”). As the heteropolyacid salt, an alkali metal salt in which a cation is an alkali metal or an ammonium salt in which a cation is an ammonium ion is known. The proton-type heteropolyacid is water-soluble, while an alkali metal salt of a heteropolyacid is generally poorly soluble because it has a large ionic radius of a cation (Non-Patent Literature 1)

As a method of producing a heteropolyacid-based catalyst, a method is known in which a slurry obtained by mixing a catalyst raw material of each element at a specific ratio so as to obtain a desired catalyst composition is produced, and the slurry and a raw material containing a cationic raw material are mixed, followed by drying or the like. For example, Patent Literature 1 describes that a catalyst is obtained by mixing a catalyst raw material liquid A containing molybdenum, phosphorus and vanadium and a catalyst raw material liquid B containing a cationic raw material to obtain a liquid containing a heteropolyacid (salt) and then drying the mixture.

Patent Literature 2 proposes a method of producing a catalyst used for producing methacrylic acid by controlling a mixing state of a slurry using a line mixer, a homomixer, a homogenizer, or the like.

### CITATION LIST

### Patent Literature

Patent Literature 1: WO 2018/037998A1

Patent Literature 1: JPH07-185354A

### Non-Patent Literature

Non-Patent Literature 1: Masayuki Ohtake, Take Onoda, Catalysis Society of Japan, Catalysts & Catalysis, vol.18, No.6 (1976), p.169

### SUMMARY OF INVENTION

### Technical Problem

According to studies by the present inventors, it was found that the particle size distribution at the time of producing a slurry has a large influence on the methacrylic acid selectivity of the obtained catalyst. However, it has been found that it is difficult to stably produce a slurry having a desired particle size distribution only by stirring and mixing. Furthermore, it has been found that, when stirring and mixing are performed using a line mixer, a homomixer, a homogenizer, or the like as described in Patent Literature 2, particles in the slurry are broken, and as a result, the performance of the obtained catalyst may be deteriorated.

Accordingly, it is an object of the present invention to provide a method of producing a catalyst used for a production of methacrylic acid which method is capable of stably producing a catalyst that enables a production of methacrylic acid with high selectivity, and a method of producing methacrylic acid using the catalyst and a method of producing methacrylic acid ester.

It is another object of the present invention to provide an apparatus for producing a catalyst used for a production of methacrylic acid which apparatus is capable of stably producing a catalyst that enables a production of methacrylic acid with high selectivity.

### Solution to Problem

As a result of diligent studies in view of the above problems, the present inventors have found that the above problem can be solved by using a catalyst produced by a specific production method as a catalyst used for a production of methacrylic acid, and has completed the present invention.

The present invention includes the following aspects of (1) to (22).

(1): A method of producing a catalyst for producing methacrylic acid by oxidizing methacrolein, comprising:
(i) preparing a slurry A1 containing a heteropolyacid containing at least phosphorus and molybdenum or a salt of the heteropolyacid containing at least phosphorus and molybdenum,
(ii) preparing a slurry A2 satisfying the following Formula (I) and Formula (II) using the slurry A1,
(iii) mixing the slurry A2 and a raw material liquid B containing a cationic raw material to prepare a slurry C, and
(iv) drying the slurry C,

660 _{A2}/α_{A1}≤0.95 (I)

α_{A2}≤D_{A2}≤50 (II)

wherein, in Formula (I), α_{A1} represents a half-value width (μm) of a particle size distribution of the slurry A, α_{A2} represents a half-value width (μm) of a particle size distribution of the slurry A2, and in Formula (II), D_{A2} represents a median diameter (&82 m) of the particle size distribution of the slurry A2.
(2): The method of producing a catalyst used for a production of methacrylic acid according to (1), which the following Formula (III):

2≤D_{C}≤50 (III)

wherein, in Formula (III), D_{C} represents a median diameter (μm) of the particle size distribution of the slurry C.
(3): The method of producing a catalyst used for a production of methacrylic acid according to (1) or (2), which the following Formula (IV):

0.6≤D_{A2}/D_{A1}<1.0 (IV)

wherein, in Formula (IV), D_{A1} represents a median diameter (μm) of the particle size distribution of the slurry A1, and D_{A2} represents a median diameter (μm) of the particle size distribution of the slurry A2.
(4): The method of producing a catalyst used for a production of methacrylic acid according to any one of (1) to (3), wherein, in the step (ii), the slurry A2 is prepared by supplying the slurry A1 to a pump.

(5): The method of producing a catalyst used for a production of methacrylic acid according to (4), wherein, when a volume of the slurry A1 prepared in the step (i) is V_{A1}, and a total volume of the slurry A1 supplied to the pump is V_{POMP}, V_{POMP}/V_{A1} at beginning of the mixing with the raw material liquid B in the step (iii) is 0.1 or more.

(6): The method of producing a catalyst used for a production of methacrylic acid according to (4) or (5), wherein V_{POMP}/V_{A1} at beginning of the mixing with the raw material liquid B in the step (iii) is 1.0 or more and 10.0 or less.
(7): The method of producing a catalyst used for a production of methacrylic acid according to any one of (4) to (6), wherein V_{POMP}/V_{A1} at beginning of the mixing with the raw material liquid B in step (iii) is larger than 1.0.

(8): The process of producing a catalyst used for a production of methacrylic acid according to any one of (4) to (7), wherein a supply speed of the slurry A1 to the pump is 1 L/min or more.

(9): The method of producing a catalyst used for a production of methacrylic acid according to any one of (4) to (8), wherein the pump is a turbo type pump or a reciprocating pump.

(10): The method of producing a catalyst used for a production of methacrylic acid according to any one of (4) to (9), wherein, when a tank for preparing the slurry A1 in the step (i) is tank 1, and a tank for mixing the slurry A2 and the raw material liquid B in the step (iii) is a tank 2, the tank 1 and the tank 2 are different tanks.

(11): The method of producing a catalyst used for a production of methacrylic acid according to (10), wherein the tank 1 and the tank 2 are connected through a pipe.

(12): The method of producing a catalyst used for a production of methacrylic acid according to (11), wherein the pump is provided in the pipe.

(13): The method of producing a catalyst used for a production of methacrylic acid according to any one of (1) to (12), wherein the cationic raw material comprises at least one selected from the group consisting of a compound containing an alkali metal and a compound containing an ammonium ion.

(14): The method of producing a catalyst used for a production of methacrylic acid according to any one of (1) to (13), wherein the slurry A1 has a viscosity of 1 to 200 cP at 30° C.

(15): The method of producing a catalyst used for a production of methacrylic acid according to any one of (1) to (14), wherein a solid content concentration of the slurry A1 is 5 to 60% by mass.

(16): The method of producing a catalyst used for a production of methacrylic acid according to any one of (1) to (15), wherein the catalyst has a composition represented by the following Formula (V):

PₐMo_{b}V_{c}Cu_{d}XₑY_{f}Z_{g}(NH₄)ₕOᵢ (V)

wherein, in the formula,

P, Mo, V, Cu, NH₄ and O represent phosphorus, molybdenum, vanadium, copper, ammonium and oxygen, respectively;

X represents at least one element selected from the group consisting of silicon, titanium, germanium, arsenic, antimony and bismuth;

Y represents at least one element selected from the group consisting of niobium, tantalum, tungsten, cerium, zirconium, silver, iron, zinc, chromium, magnesium, cobalt, manganese, barium and lanthanum;

Z represents at least one element selected from the group consisting of lithium, sodium, potassium, rubidium and cesium;

a to i represent molar ratios of each component, and

when b is 12, a=0.5 to 3, c=0.01 to 3, d=0.01 to 2, e=0.1 to 3, f=0 to 3, g=0.01 to 3, h=0 to 20, and i represents a molar ratio of oxygen required to satisfy a valence of each of the components.

(17): A method of producing methacrylic acid, comprising oxidizing methacrolein in a presence of the catalyst produced by the method according to any one of (1) to (16).

(18): A method of producing a methacrylic ester, comprising esterifying the methacrylic acid produced by the method according to (17).

(19): An apparatus for producing a catalyst used for producing methacrylic acid by oxidizing methacrolein, comprising:
a tank for preparing a slurry A1 containing at least phosphorus and molybdenum,
a means for preparing a slurry A2 satisfying the following Formula (I) and Formula (II) using the slurry A1:

α_{A2}/α_{A1}≤0.95 (I)

2≤D_{A2}≤50 (II)

wherein, in Formula (I), am represents a half-value width (μm) of a particle size distribution of the slurry A1, α_{A2} represents a half-value width (μm) of a particle size distribution of the slurry A2, and in Formula (II), DA2 represents a median diameter (μm) of the particle size distribution of the slurry A2.

(20): The apparatus according to (19), further comprising tank 1 for preparing the slurry A1 and tank 2 for mixing the slurry A1 with a raw material liquid B containing a cationic raw material.

(21): The apparatus for producing a catalyst used for a production of methacrylic acid according to (20), wherein the tank 1 and the tank 2 are connected through a pipe.

(22): The apparatus for producing a catalyst used for a production of methacrylic acid according to (21), wherein a pump is provided in the pipe.

### Advantageous Effects of Invention

According to the present invention, it is possible to provide a method of producing a catalyst used for a production of methacrylic acid which method is capable of stably producing a catalyst that enables a production of methacrylic acid with high selectivity, and a method of producing methacrylic acid using the catalyst and a method of producing of methacrylic ester. Furthermore, it is possible to provide an apparatus for producing a catalyst used for a production of methacrylic acid which apparatus is capable of stably producing a catalyst that enables a production of methacrylic acid with high selectivity.

### BRIEF DESCRIPTION OF DRAWINGS

FIG. 1 is a schematic view of a manufacturing apparatus according to an embodiment of the present invention.

### DESCRIPTION OF EMBODIMENTS

Hereinafter, embodiments of the present invention will be described in detail, but the description of the constituent elements described below is an example of an embodiment of the present invention, and the present invention is not limited to these contents.

### Catalyst Used for Production of Methacrylic Acid

A catalyst produced by a production method according to an embodiment of the present invention is used in oxidizing methacrolein to produce methacrylic acid. From the viewpoint of improving selectivity in the production of methacrylic acid, the catalyst preferably has a composition represented by the following formula (V). In embodiments of the present invention, when the catalyst is formed using a carrier, the catalyst means one containing the carrier, and the following Formula (V) is a composition in consideration of the carrier.

PₐMo_{b}V_{c}Cu_{d}XₑY_{f}Z_{g}(NH₄)ₕOᵢ (V)

(In the formula,

P, Mo, V, Cu, NH₄ and O represent phosphorus, molybdenum, vanadium, copper, ammonium and oxygen, respectively;

X represents at least one element selected from the group consisting of silicon, titanium, germanium, arsenic, antimony and bismuth;

Y represents at least one element selected from the group consisting of niobium, tantalum, tungsten, cerium, zirconium, silver, iron, zinc, chromium, magnesium, cobalt, manganese, barium and lanthanum;

Z represents at least one element selected from the group consisting of lithium, sodium, potassium, rubidium and cesium;

a to i represent molar ratios of each component, and

when b is 12, a=0.5 to 3, c=0.01 to 3, d=0.01 to 2, e=0.1 to 3, f=0 to 3, g=0.01 to 3, h=0 to 20, and i represents a molar ratio of oxygen required to satisfy a valence of each of the components.)

Note that the molar ratio of each element is a value calculated by analyzing components in which the catalyst is dissolved in ammonia water by an ICP emission analysis method. In addition, the molar ratio of ammonium is set to a value calculated by analyzing the catalyst by the Kjeldahl method.

Method for Producing Catalyst used for Production of Methacrylic Acid

A method of producing a catalyst for producing methacrylic acid by oxidizing methacrolein according to an embodiment of the present invention, includes the following steps (i) to (iv).

(i) Preparing a slurry A1 containing a heteropolyacid containing at least phosphorus and molybdenum or a salt of the heteropolyacid containing at least phosphorus and molybdenum.

(ii) Preparing a slurry A2 satisfying the following Formula (I) and Formula (II) using the slurry A1.

(iii) Mixing the slurry A2 and a raw material liquid B containing a cationic raw material to prepare a slurry C.

(iv) Drying the slurry C.

α_{A2}/α_{A1}≤0.95 (I)

2≤D_{A2}≤50 (II)

(In Formula (I), α_{A1} represents a half-value width (μm) of a particle size distribution of the slurry A1, α_{A2} represents a half-value width (μm) of a particle size distribution of the slurry A2, and in Formula (II), D_{A2} represents a median diameter (μm) of the particle size distribution of the slurry A2.)

Hereinafter, each step will be described in detail.

### (Step (i))

In step (i), the slurry A1 containing a heteropolyacid (salt) comprising at least phosphorus and molybdenum is prepared.

When the slurry A1 contains at least these elements, a catalyst having a higher methacrylic acid selectivity can be produced.

The slurry A1 may contain other elements. For example, it may contain V (vanadium) or Cu (copper) in the above Formula (V), and may also contain an X element or a Y element.

Note that other elements other than phosphorus and molybdenum in Formula (V) can also be added in the steps after the step (i).

The slurry A1 can be prepared by dissolving or suspending a raw material compound of a catalyst component containing at least phosphorus and molybdenum in a solvent.

### <Raw Material Compound of Catalyst Component>

The raw material compound of the catalyst component is not particularly limited, and nitrates, carbonates, acetates, ammonium salts, oxides, halides, oxoacids, oxoacid salts, and the like of each constituent element of the catalyst can be used alone or in combination of two or more.

Examples of the raw material compound of molybdenum include molybdenum oxide such as molybdenum trioxide, ammonium molybdate such as ammonium paramolybdate and ammonium dimolybdate, and molybdenum chloride.

Examples of the raw material compound for phosphorus include, for example, phosphoric acid, phosphorous pentoxide, and ammonium phosphate.

When producing a catalyst further containing vanadium in addition to phosphorus and molybdenum, examples of the raw material compound of vanadium include ammonium metavanadate, vanadium pentoxide, vanadium chloride, and vanadyl oxalate.

When producing a catalyst further containing copper in addition to phosphorus and molybdenum, examples of the raw material compounds of copper include copper sulfate, copper nitrate, copper oxide, copper carbonate, copper acetate, copper chloride, and the like.

As the raw material compound of the catalyst component, one kind thereof may be used for each element constituting the catalyst component, or two or more kinds thereof may be used in combination.

The concentration of the raw material compound of the catalyst component in the slurry A1 is not particularly limited, but is preferably set within a range of 5% by mass or more and 90% by mass or less.

### <Solvent>

Examples of the solvent include water, ethyl alcohol, acetone and the like. These may be used alone or in combination of two or more. Among these, it is preferable to use water from an industrial viewpoint.

### <Formation of Heteropolyacid (Salt)>

It is preferable that the slurry A1 is prepared by adding a raw material compound of a catalyst component to a solvent using a preparation vessel and stirring while heating. The heating can usually be carried out in the range of 30 to 150° C., and is preferably carried out in the range of 60 to 150° C. By setting the heating temperature to 60° C. or higher, the rate of formation of the heteropolyacid (salt) can be sufficiently increased, and by setting the temperature to 150° C. or lower, evaporation of the solvent can be suppressed. The lower limit of the heating temperature is more preferably 80° C. or higher, even more preferably 90° C. or higher. The upper limit of the heating temperature is more preferably 130° C. or less, even more preferably 110° C. or less. In addition, depending on the vapor pressure of the solvent used, it may be concentrated or refluxed at the time of heating, or may be heated under pressurized conditions by operating in a sealed vessel.

The temperature rising rate is not particularly limited, but is preferably 0.8 to 15° C/min. By the temperature rising rate is 0.8° C./min or more, it is possible to shorten the time required for step (i). Further, by the temperature rising rate is 15° C./min or less, the temperature can be raised by using a normal temperature raising facility.

The stirring is preferably performed at a stirring power 0.01 kW/m³ or higher, more preferably at a 0.05 kW/m³ or higher. By setting the stirring power to 0.01 kW/m³ or more, local unevenness of the temperature and the component in the slurry A1 can be reduced, and a structure suitable as a catalyst for producing methacrylic acid can be stably formed. In addition, from the viewpoint of production cost of the catalyst, the stirring is preferably performed under normal stirring power 3.5 kW/m³ or less.

### <Particle Size Distribution of Slurry A1>

The median diameter (DA') of the particle size distribution of the slurry A1 is not particularly limited, but is preferably 2 to 50 μm. Thus, it is possible to easily prepare the slurry A2 having a predetermined particle size distribution in step (ii) described later. The lower limit of D_{A1} is more preferably 2.5 μm or more. Further, the upper limit of D_{A1} is more preferably 25μm or less, and still more preferably 10 μm or less. In this specification, the median diameter indicates a particle diameter corresponding to a cumulative 50% by volume in a volume-based particle size distribution measured by a laser diffraction type particle size distribution measurement method.

The half-value width (α_{A1}) of the particle size distribution of the slurry A1 obtained in step (i) is not particularly limited, but is preferably 3 to 10 μm, and more preferably 5 μm or more, and particularly when the half-value width (α_{A1}) is 5 μm or more, the effect according to an embodiment of the present invention can be effectively obtained. In this specification, the half-value width indicates a peak width at half the height of the peak having the largest particle diameter in the volume-based particle size distribution measured by the laser diffraction type particle size distribution measurement method. It should be noted that the peak means that the maximum frequency is 0.5% or more.

### <Physical Properties of Slurry A1>

The pH of the slurry A1 is not particularly limited, but is preferably 0.1 to 4, and the lower limit of the pH is preferably 0.5 or more and the upper limit of the pH is more preferably 3 or less.

When the pH of the slurry A1 is 0.1 or more, the step of mixing the raw material liquid B in the step (iii) described later can be stably performed. Further, when the pH of the slurry A1 is 4 or less, a reaction for producing the heteropolyacid (salt) suitable for methacrylic acid production is stabilized. As a method of setting the pH of the slurry A1 to 0.1 to 4, for example, a method of using molybdenum trioxide as a molybdenum raw material or a method of appropriately selecting a raw material compound and adjusting the content of nitrate ions or oxalate ions can be mentioned.

The viscosity of the slurry A1 is not particularly limited, but is preferably from 1 to 200 cP at 30° C. When the viscosity of the slurry A1 at 30° C. is 1 cP or more, the step (ii) described later can be stably performed, and when it is 200 cP or less, mixing the slurry A1 with the raw material liquid B in the step (iii) described later becomes good. The lower limit of the viscosity of the slurry A1 at 30° C. is more preferably 5 cP or more, and still more preferably 10 cP or more. Further, the upper limit of the viscosity is more preferably 150 cP or less, and still more preferably 100 cP or less. The viscosity of the slurry A1 can be measured by a method using a B-type viscometer described later.

The specific gravity of the slurry A1 is not particularly limited, but is preferably 1.05 to 1.25 kg/L from the viewpoint of stably performing step (ii) described later. The solid content concentration of the slurry A1 (the mass ratio of the solid content to the entire slurry A1) is not particularly limited, but is preferably 5 to 60% by mass. Thus, in step (iii) described later, the slurry C can be stably prepared. The lower limit of the solid content concentration of the slurry A1 is more preferably 10% by mass or more, and still more preferably 15% by mass or more. Further, the upper limit of the solid content concentration is more preferably 55% by mass or less, and still more preferably 50% by mass or less.

<Volume of Slurry A1>

Considering industrial production, the volume of the slurry A1 is preferably 0.2 m³ or more in total with the raw material liquid B from the viewpoint of manufacturing cost, and is more preferably 0.8 m³ or more, and still more preferably 1.5 m³ or more. The upper limit of the volume is not particularly limited, but can be set to, for example, 5 m³ or less.

(Step (ii))

In step (ii), the slurry A2 satisfying the following Formulas (I) and (II) is prepared using the slurry A1 obtained in step (i). The slurry A2 contains the heteropolyacid (salt) produced in the step (i).

α_{A2}/α_{A1}≤0.95 (I)

2≤D_{A2}≤50 (II)

In Formula (I), am represents a half-value width (μm) of a particle size distribution of the slurry A1, α_{A2} represents a half-value width (μm) of a particle size distribution of the slurry A2. α_{A2}/α_{A1} is the ratio of the half-value widths of the particle size distributions of the slurry A1 and the slurry A2, when α_{A2}/α_{A1} is less than 1, aggregated particles in the slurry A1 are dispersed, indicating that the slurry A2 containing more uniform particles is prepared. In Formula (II), D_{A2} represents a median diameter (μm) of the particle size distribution of the slurry A2.

By preparing the slurry A2 satisfying the above Formulas (I) and (II), a catalyst having a high methacrylic acid selectivity can be obtained. It is considered that this is because by producing a catalyst using the slurry A2 in which the aggregated particles in the slurry A1 are dispersed (disaggregated), the slurry A2 having a defined median diameter, a heteropolyacid salt suitable for methacrylic acid production is produced in the step (iii) described later. The slurry A2 satisfies α_{A2}/α_{A1}≤0.95, it is preferable to satisfy α_{A2}/α_{A1}≤0.9. The lower limit of α_{A2}/α_{A1} is not particularly limited, but a sufficient effect can be obtained at 0.7 or more (α_{A2}/α_{A1}≥0.7). Further, α_{A2} is preferably 9 μm or less, more preferably 8 μm or less, and still more preferably 7 μm or less.

The lower limit of D_{A2} is 2 μm or more, and is preferably 2.5 μm or more. The upper limit of D_{A2} is 50 μm or less, preferably 25 μm or less, and more preferably 10 μm or less.

Further, it is preferable that the slurry A2 satisfies the following Formula (IV).

0.6≤D_{A2}/D_{A1}<1.0 (IV)

In Formula (IV), D_{A1} represents a median diameter (μm) of the particle size distribution of the slurry A1, and D_{A2} represents a median diameter (μm) of the particle size distribution of the slurry A2. D_{A2}/D_{A1} is the ratio of the median diameters of the particle size distributions of the slurry A1 and the slurry A2, when D_{A2}/D_{A1} is less than 1, aggregated particles in the slurry A1 are dispersed (disaggregated), indicating that the median diameter of the slurry A2 is reduced. On the other hand, when the particles in the slurry A1 are broken and D_{A2}/D_{A1} becomes less than 0.6, the methacrylic acid selectivity of the obtained catalyst decreases. It is more preferable that the slurry A2 satisfies 0.7≤D_{A2}/D_{A1}<1.0.

The slurry A2 satisfying the Formulas (I) and (II) can be prepared using a tank for preparing the slurry A1 and an apparatus for producing a catalyst provided with means for preparing the slurry A2 satisfying the Formulas (I) and (II) using the slurry A1. The means for preparing the slurry A2 satisfying the above Formulas (I) and (II) is not particularly limited, but examples thereof include a method of preparing the slurry A2 in which the slurry A1 is supplied to a pump to use a shearing force, a method of preparing the slurry A2 in which the slurry A1 is directly subjected to vibration by irradiating the particles with ultrasonic waves, or a method in which the aggregated particles in the slurry A1 are separated using a sieve (filtration), gravity, inertia, centrifugal force, or the like.

Hereinafter, a method of preparing the slurry A2 by supplying the slurry A1 to the pump will be described with reference to the accompanying drawings and illustrate an embodiment.

### <Apparatus for Producing Catalyst Used for Producing Methacrylic Acid>

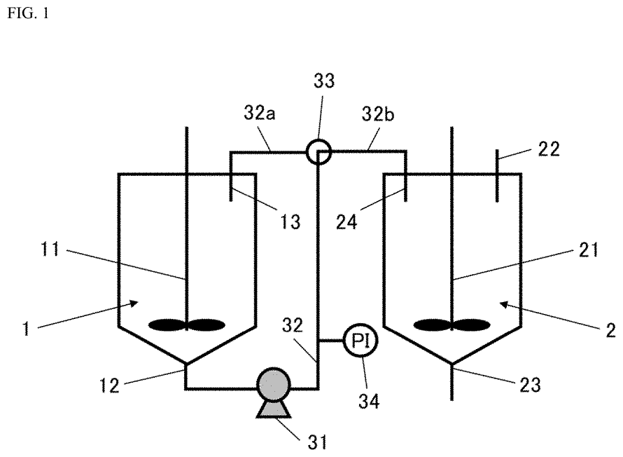

The method of preparing the slurry A2 by supplying the slurry A1 to the pump is not particularly limited, and for example, it can be performed using a manufacturing apparatus as shown in FIG. 1 (hereinafter, also simply referred to as “the present manufacturing apparatus”).

The manufacturing apparatus shown in FIG. 1 has a tank 1 and a tank 2, the tank 1 and the tank 2 is connected through a pipe 32 provided with a pump 31. The tank 1 is provided with an agitator 11, an outlet 12, and a liquid return port 13. The tank 2 is provided with an agitator 21, a feed port 22, an outlet 23, and a liquid feed port 24.

The pipe 32 is connected to the tank 1 through the outlet 12 and the liquid return port 13 of the tank 1, and is connected to the tank 1 through the liquid feed port 24 of the tank 2. The pipe 32 includes a pipe portion 32a leading to the liquid return port 13 of the tank 1, and a pipe portion 32b leading to the liquid feed port 24 of the tank 2, the branch portion of the pipe portion 32a and the pipe portion 32b, the two-way valve 33 is provided. By the two-way valve 33, the slurry A1 sent from the pump 31 can be switched so that the slurry A1 can be sent to either the return liquid port 13 or the liquid feed port 24. Further, by attaching the pressure gauge 34 between the pump 31 and the two-way valve 33, it is possible to measure the discharge pressure of the pump 31. The manufacturing apparatus shown in FIG. 1 is an example, and may be provided with other configurations.

The volumes of the tank 1 and the tank 2 are not particularly limited, and may be appropriately selected according to the volume of the slurry A1. Further, the materials of the tank 1 and the tank 2 are not particularly limited, and a tank made of stainless steel or a tank coated with glass on the inside can be used.

The type of pump 31 is not particularly limited, and commonly used turbo-type pumps, positive displacement pumps, and the like can be used. Examples of the turbo type pumps include centrifugal pumps, propeller pumps (axial flow pumps, mixed flow pumps), viscous pumps, and the like. Examples of the positive displacement pumps include reciprocating pumps, rotary pumps, and the like. Among these, a turbo type pump or a reciprocating type pump is preferably used, and a turbo type pump is more preferably used.

The inner diameter of the pipe 32 (including the pipe portions 32a and 32b) is not particularly limited, but is preferable 5 to 500 mm from the viewpoint of the processing amount in consideration of industrial production. The lower limit of the inner diameter of the pipe 32 is more preferably 7 mm or more, and still more preferably 10 mm or more. Further, the upper limit of the inner diameter is more preferably 200 mm or less, even more preferably 100 mm or less.

### <Preparation of Slurry A2 by Supplying Slurry A1 to the Pump>

Using the present manufacturing apparatus described above, it is possible to prepare the slurry A2 satisfying the Formulas (I) and (II) in the following manner.

First, the slurry A1 is prepared in the tank 1. At this time, the tank 1 may be used as the preparation vessel in the step (i), or the slurry A1 prepared by the step (i) may be supplied to the tank 1. In addition, at this time, the slurry A1 may be agitated by the agitator 11

After that, the slurry A1 is drawn out from the outlet 12, supplied to the pump 31 via the pipe 32, and fed from the liquid feed port 24 via the pipe portion 32b to the tank 2. At this time, by applying a shearing force by the pump 31 to the slurry A1, aggregated particles in the slurry A1 are dispersed, and the slurry A2 containing more uniform particles can be prepared.

In the liquid sending of the slurry A1, the slurry A1 may be circulated by being sent back to the tank 1 from the liquid return port 13 via the pipe portion 32a. The liquid sending of the slurry A1 to the tank 2 and the liquid sending of the slurry A1 to the tank 1 may be performed in combination of both. That is, after supplying the slurry A1 to the pump 31, the slurry A1 may be sent back to the tank 1, and at least a part of the slurry may be circulated, and then the slurry A1 may be sent from the tank 1 to the tank 2 again using the pump 31. Here, “circulation” means that the slurry A1 drawn out from the tank 1 and supplied to the pump 31 is returned to the tank 1 again, and the circulation is a kind of liquid sending.

The case where the slurry A1 drawn out from the outlet 12 of the tank 1 is sent to the liquid return port 13 side of the tank 1 and the case where the slurry A1 is sent to the liquid feed port 24 side of the tank 2 can be controlled by switching the liquid feeding line of the slurry A1 (pipe portions 32a, 32b) using the two-way valve 33.

In addition, the liquid sending of the slurry A1 may be performed while agitating with the agitators 11 and 21 in the tank 1 and/or the tank 2.

The supply speed of the slurry A1 to the pump 31 is not particularly limited, but is preferably 1 L/min or more. Accordingly, a shearing force for dispersing the aggregated particles in the slurry A1 can be generated, and the slurry A2 can be efficiently prepared. Further, the supply speed of the slurry A1 to the pump 31 is preferably 400 L/min or less. Thus, it is possible to prevent the particles in the slurry A1 from being broken by applying an excessive shearing force. The lower limit of the supply speed of the slurry A1 to the pump 31 is more preferably 10 L/min or more, still more preferably 100 L/min or more, and particularly preferably 150 L/min or more. Further, the upper limit of the supply speed is more preferably 300 L/min or less, and still more preferably 250 L/min or less.

It is preferable to start mixing with the raw material solution B in the step (iii) described later when V_{POMP}/V_{A1} is 0.1 or more, where the volume of the slurry A1 prepared in the step (i) is V_{A1} and the total volume of the slurry A1 supplied to the pump 31 is V_{POMP}. Thus, a heteropolyacid salt suitable for methacrylic acid production can be stably produced in step (iii). If at least a part of the slurry A1 is supplied to the pump 31 and the prepared slurry A2 is present in the tank 2, mixing with the raw material liquid B in the tank 2 may be started while supplying the remaining slurry A1 to the pump 31. The lower limit of V_{POMP}/V_{A1} is more preferably 0.5 or more, still more preferably 1.0 or more, particularly preferably larger than 1.0, and most preferably 2.0 or more. Here, the fact that V_{POMP}V_{A1} is greater than 1.0 means that the slurry A1 is supplied to the pump 31, then sent back to the tank 1, and at least a part of the slurry A1 is circulated, and thereafter, using the pump 31 again, the slurry A1 is sent from the tank 1 to the tank 2 (as a result, a slurry containing the slurry that is circulated and sent again is mixed with the raw material B). On the other hand, in order to prevent the particles in the slurry A1 from being destroyed due to coming into contact with each other, the upper limit of V_{POMP}/V_{A1} is preferably 10.0 or less, more preferably 5.0 or less, and still more preferably 4.0 or less. It is preferable that V_{POMP}/V_{A1} is appropriately adjusted according to the supply speed of the slurry A1 to the pump 31. When the supply speed of the slurry A1 is high, the shearing force is high and accordingly the aggregated particles in the slurry A1 are easily dispersed. Therefore, even when V_{POMP}/V_{A1} is small, the slurry A2 can be easily prepared.

The temperature during feeding of the slurry A1 is not particularly limited, but is preferable a temperature at which the solvent is not vaporized and no cavitation is generated in the pump, and above all, in order to stabilize the properties of the slurry A1, the temperature is preferably 30 to 150° C. The lower limit of the temperature during feeding of the slurry A1 is more preferably 40° C. or higher, and still more preferably 50° C. or higher. Further, the upper limit of the temperature is more preferably 120° C. or less, more preferably 100° C. or less.

The discharge pressure of the pump 31 for supplying the slurry A1 is not particularly limited, but is preferable 1 to 1000kPa in order to stabilize the properties of the slurry A1. The lower limit of the discharge pressure of the pump 31 is more preferably 10 kPa or more, still more preferably 100 kPa or more, and the upper limit of the discharge pressure is more preferably 800 kPa or less, and still more preferably 600 kPa or less.

(Step (iii))

In the step (iii), the slurry A2 obtained in step (ii) and the raw material liquid B containing a cationic raw material are mixed to prepare the slurry C.

By mixing the slurry A2 and the raw material liquid B, a counter cation of the heteropolyacid (salt) contained in the slurry A2 is replaced with a cation contained in the raw material liquid B, and the slurry C containing the heteropolyacid salt is obtained.

By mixing the slurry A2 satisfying the above Formulas (I) and (II) with the raw material liquid B to produce the slurry C, a catalyst having a high selectivity in methacrylic acid production can be obtained. Although this mechanism is not clear, the following reasons are conceivable.

As described above, the slurry A1 contains the heteropolyacid (salt) containing at least phosphorus and molybdenum, and particles in the slurry A1 containing this heteropolyacid (salt) are usually present in an aggregated state. When the raw material liquid B is mixed with the slurry A1 in this state to produce the slurry C, since the raw material liquid B does not easily reach inside the aggregated particles in the slurry A1 uniformly, it is difficult to uniformly form the heteropolyacid salt. However, in the step (ii) described above, the aggregated particles in the slurry A1 are dispersed (disaggregated), and the slurry A2 containing more uniform particles is prepared. By mixing the slurry A2 and the raw material liquid B, the heteropolyacid salt can be uniformly formed. It is considered that this uniform heteropolyacid salt is suitable as a catalyst for producing methacrylic acid, and as a result, a catalyst having a high methacrylic acid selectivity can be obtained.

Note that, when the slurry A1 and the raw material liquid B are mixed and then a processing for dispersing the formed particles is performed, or when a processing for dispersing only the particles in the raw material liquid B is performed, it is difficult to obtain a catalyst having a high methacrylic acid selectivity as described above. Thus, it is extremely important to first disperse the aggregated particles in the slurry A1 to prepare the slurry A2, and then mix the slurry A2 with the raw material liquid B. However, if necessary, a processing for dispersing particles in the raw material liquid B may be performed.

### <Preparation of Raw Material Liquid B>

The raw material liquid B contains a cationic raw material. The raw material liquid B can be prepared by dissolving or suspending the cationic raw material in a solvent.

Here, the “cationic raw material” includes at least one selected from the group consisting of a compound containing an alkali metal, a compound containing an alkaline earth metal, a compound containing a transition metal, a compound containing a base metal, and a compound containing nitrogen (including ammonia, a compound containing ammonium ion or an alkylammonium ion, or a nitrogen-containing heterocyclic compound). Examples of the alkali metal include lithium, sodium, potassium, rubidium, and cesium. Examples of the alkaline earth metal include magnesium, calcium, strontium, and barium. Examples of the compound containing an alkali metal, the compound containing an alkaline earth metal, the compound containing a transition metal, and the compound containing a base metal include nitrates of an alkali metal, an alkaline earth metal, a transition metal or a base metal, carbonates thereof, bicarbonates thereof, acetates thereof, sulfates thereof, ammonium salts thereof, oxides thereof, hydroxides thereof, halides thereof, oxoacids thereof, and oxoacid salts thereof. Examples of the compound containing ammonium ion include ammonium bicarbonate, ammonium carbonate, ammonium nitrate, ammonium phosphate, and ammonium vanadate. Examples of the compound containing an alkylammonium ion include halides or hydroxides of tetramethylammonium, tetraethylammonium, tetran-propylammonium, tetran-butylammonium, triethylmethylammonium and the like. Examples of the nitrogen-containing heterocyclic compound include pyridine, piperidine, piperazine, pyrimidine, quinoline, isoquinoline, and alkyl derivatives thereof. These may be used alone or in combination of two or more. Among these, from the viewpoint of obtaining a catalyst for producing methacrylic acid having a higher methacrylic acid selectivity, as the cationic raw material, at least one selected from the group consisting of the compound containing an alkali metal and the compound containing an ammonium ion is preferable, and the compound containing an alkali metal and the compound containing an ammonium ion are more preferable.

Examples of the solvent include water, ethyl alcohol, and acetone. These may be used alone or in combination of two or more. Among these, it preferable to use water.

When a plurality of types of the cationic raw material are used, using a plurality of preparation vessels and each cationic raw material is dissolved or suspended in a solvent, thereby a plurality of raw material liquids B may be prepared as in the raw material solutions B1, B2, . . .

The concentration of the cationic raw material in the raw material liquid B is not particularly limited, but is preferably set within the range of 5 to 90% by mass.

When the raw material liquid B contains particles based on the above raw material, the median diameter of the particle size distribution of the raw material liquid B is not particularly limited, but is preferably 5 μm or less. Thereby, the slurry C satisfying Formula (III) described later can be easily prepared. The upper limit of the median diameter of the particle size distribution of the raw material liquid B is more preferably 3 μm or less, and still more preferably 1 μm or less. The raw material liquid B is preferably in a solution state in which all of the raw materials are dissolved, and when particles based on the above raw material are contained, the upper limit of the median diameter is preferably small as described above. However, from the viewpoint of being able to be used as a nucleus for particle generation of a reactant, particles having a median diameter of 0.01 μm or more may be present, and particles having a median diameter of 0.05 μm or more may be present, and further particles having a median diameter of 0.1 μm or more may be present.

### <Mixing of Slurry A2 and Raw Material Liquid B>

In the mixing of the slurry A2 and the raw material liquid B, one of the slurry A2 and the raw material liquid B can be added to the other liquid and mixed to prepare the slurry C. In other words, the raw material liquid B is added to the slurry A2 and mixed, or the slurry A2 is added to the raw material liquid B and mixed.

When a plurality of raw material liquids B1, B2, . . . are prepared using a plurality of preparation vessels, the raw material liquids B1, B2, . . . may be added to the slurry A2 in no particular order, or may be added at the same time. Also, the slurry A2 may be added to any one of the raw material liquids B, and the obtained liquid and the other raw material liquid(s) B may be mixed. After the slurry A2 is divided into a plurality of portions and added to each of the raw material liquids B, each of the obtained liquids may be mixed.

In the mixing described above, it is preferable to add the raw material liquid B to the slurry A2 and mix them, and specifically, it is preferable to add the raw material liquid B in a tank containing the slurry A2 and mix them. For example, when using the manufacturing apparatus shown in FIG. 1, the slurry A2 can be prepared by supplying the slurry A1 in the tank 1 to the pump 31, and transferred to the tank 2, and then the raw material liquid B can be added from the feed port 22 to the slurry A1 and mixed with it. It is presumed that by mixing the raw material liquid B containing the cationic raw material as an additive liquid, particles which are more effective for improving the methacrylic acid selectivity can be easily generated.

The temperature at which the slurry A2 and the raw material liquid B are mixed is not particularly limited, but is preferably 30 to 150° C. When the temperature is 30° C. or higher, the heteropolyacid salt can be stably produced. When the temperature is 150° C. or lower, evaporation of the solvent can be avoided to produce the heteropolyacid salt in a stable environment. The lower limit of this temperature is preferably 40° C. or higher, and the upper limit is more preferably 100° C. or lower.

When mixing the slurry A2 and the raw material liquid B, agitating may be performed. Examples of agitating device include a known agitating device such as a rotary blade agitator, a rotary agitator, a pendulum type linear motion agitator, a shaker which shakes the entire vessel, and a vibration type agitator using ultrasonic waves or the like.

<Slurry C>

The slurry C obtained by mixing the slurry A2 and the raw material liquid B preferably satisfies the following Formula (III).

2≤D_{C}≤50 (III)

In Formula (III), Dc represents a median diameter (μm) of the particle size distribution of the slurry C.

When the slurry C satisfies the Formula (III), pores suitable for methacrylic acid production can be formed.

The lower limit of the median diameter Dc of the particle size distribution of the slurry C is preferably 2.5 μm or more, and more preferably 3 μm or more. The upper limit of Dc is preferably 50 μm or less, more preferably 25 μm or less, and still more preferably 10 μm or less.

The half-value width ac of the particle size distribution of the slurry C is preferably 10 μm or less, more preferably 9 μm or less, still more preferably 8 μm or less, and particularly preferably 7.5 μm or less.

The slurry C contains the above-mentioned slurry A1 and a metal and the like mentioned in the raw material liquid B, and from the viewpoint of improving selectivity in methacrylic acid production, it is preferable that the component after drying the slurry C has the composition represented by the above Formula (V). A raw material compound for an element may be added to the slurry A1 or the raw material liquid B, or after mixing of the slurry A2 and the raw material liquid B so that the component has the composition represented by the above Formula (V).

Regarding the slurry C, which contains the heteropolyacid salt, it is preferable that this heteropolyacid salt has a Keggin type structure. When the slurry C contains the heteropolyacid salt having a Keggin-type structure, the generated particles are less likely to change and can be stably present, so that a catalyst having a high methacrylic acid selectivity can be obtained. As a method of obtaining the slurry C containing the heteropolyacid salt having a Keggin type structure, for example, in the above-mentioned step (i), a method in which the pH of the slurry A1 is adjusted to be low in advance and the pH of the slurry C is set to 4 or less, preferably 3 or less is mentioned. The pH of the slurry C can be set in a range of 0.1 to 4, and the lower limit is preferably 0.5 or more, more preferably 1 or more, and the upper limit is preferably 3 or less. The inclusion of the heteropolyacid salt having a Keggin-type structure in the slurry C can be confirmed by measuring the dried slurry C by infrared absorption analysis. When containing the heteropolyacid salt having a Keggin-type structure, the resulting infrared absorption spectrum has characteristic peaks around 1060, 960, 870, and 780 cm⁻¹.

(Step (iv))

In the step (iv), the slurry C obtained in step (iii) is dried to obtain a dried product. Examples of the drying method include known methods such as a drum drying method, an air flow drying method, an evaporation-drying method, and a spray drying method. Among these, it is preferable to use a spray drying method because a particulate dried product can be obtained and the dried product has a well-shaped spherical shape.

The drying temperature varies depending on the drying method, but the drying can be usually performed at 100 to 500° C., and the lower limit of the drying temperature is preferably 140° C. or higher, and the upper limit of the drying temperature is preferably 400° C. or lower.

The drying is preferably performed so that the moisture content of the obtained dried product is 4.5% by mass or less, and more preferably 0.1 to 4.5% by mass.

These conditions are not particularly limited, and can be appropriately selected depending on the shape and size of the desired dried product.

The dried product obtained in step (iv) exhibits catalytic performance and can be used as a catalyst for producing methacrylic acid, and it is preferable to performing molding or calcination described later because the performance as a catalyst is improved. In the present invention, the products including those after molding and after calcination are collectively referred to as catalysts.

### (Molding Step)

In the molding step, the dried product obtained in the step (iv) is molded as necessary to obtain a molded article. The molding may be performed after the calcination described later.

The molding method is not particularly limited, and known dry and wet molding methods can be applied, and examples thereof include tableting molding, press molding, extrusion molding, and granulation molding. The shape of the molded article is not particularly limited, and examples thereof include a columnar shape, a ring shape, a spherical shape. At the time of molding, it is preferable to mold only the dried product without adding a carrier or the like thereto, but if necessary, a known additive such as graphite or talc may be added. When a carrier is used, the carrier is not particularly limited, but silica is preferable.

### (Calcination Step)

It is preferable to calcine the dried product obtained in the step (iv) and the molded article obtained in the molding step from the viewpoint of methacrylic acid selectivity.

The calcination can be performed under the flow of at least one of an oxygen-containing gas such as air and an inert gas, and preferably under an oxygen-containing gas flow such as air. Here, the inert gas refers to a gas which does not reduce the catalytic activity, and examples thereof include nitrogen, carbon dioxide gas, helium, and argon. Only one kind of these may be used, or two or more kinds thereof may be mixed and used.

The shape of the calcination container is not particularly limited, but a box-shaped or tubular container can be used. Further, the dried product or molded article can be divided into a plurality of containers, filled and calcined. Among them, it is preferable to use a tubular container having a cross-sectional area of 1 to 100 cm².

The calcination temperature (maximum temperature at the time of calcination) is preferably 200 to 700° C., the lower limit is more preferably 320° C. or higher, and the upper limit is more preferably 450° C. or lower.

As described above, a catalyst for producing methacrylic acid can be produced.

### Method of Producing Methacrylic Acid

In a method of producing methacrylic acid according to an embodiment of the present invention, methacrylic acid is produced by oxidizing methacrolein in the presence of a catalyst used for producing methacrylic acid produced by the above-described method. According to this method, methacrylic acid can be produced with high selectivity.

Specifically, methacrylic acid can be produced by bringing a raw material gas containing methacrolein and oxygen into contact with the above-mentioned catalyst used for producing methacrylic acid. The reaction can usually be carried out in a fixed bed. The catalyst layer may be one layer, or two or more layers. The catalyst used for producing methacrylic acid may be a mixture of other additives.

The concentration of methacrolein in the raw material gas is not particularly limited, but is preferably 1 to 20% by volume, the lower limit is more preferably 3% by volume or more, and the upper limit is more preferably 10% by volume or less. The components other than methacrolein contained in the raw material gas are not particularly limited, and examples thereof include water, oxygen, and nitrogen. In addition, methacrolein may contain a small amount of impurities such as lower saturated aldehyde which do not substantially affect the present reaction.

The concentration of oxygen in the raw material gas is preferably 0.4 to 4 mol with respect to 1 mol of methacrolein, the lower limit is more preferably 0.5 mol or more with respect to 1 mol of methacrolein, and the upper limit is more preferably 3 mol or less with respect to 1 mol of methacrolein. As an oxygen source, air is preferable from the viewpoint of economical efficiency.

If necessary, a gas or the like enrich with oxygen by adding pure oxygen to air may be used.

The raw material gas may be a gas obtained by diluting methacrolein and oxygen (or an oxygen source) with an inert gas such as nitrogen or carbon dioxide gas. Further, water vapor may be added to the raw material gas. By carrying out the reaction in the presence of water vapor, methacrylic acid can be obtained at a higher selectivity. The concentration of water vapor in the raw material gas is preferably 0.1 to 50% by volume, the lower limit is more preferably 1% by volume or more, and the upper limit is more preferably 40% by volume or less. The contact time between the raw material gas and the catalyst used for producing methacrylic acid is preferably 0.1 to 30 seconds, the lower limit is more preferably 1 seconds or more, and the upper limit is more preferably 10 seconds or less.

The reaction pressure is preferably 0.1 to 1MPa (G) or less. Not that (G) means that it is a gauge pressure.

The reaction temperature is not particularly limited, but is preferably 200 to 450° C., the lower limit is more preferably 250° C. or higher, and the upper limit is more preferably 400° C. or lower.

### Method of Producing Methacrylic Acid Ester

The method of producing a methacrylic ester according to an embodiment of the present invention includes esterifying methacrylic acid produced by the above-described method. According to the method, methacrylic acid esters can be obtain by using methacrylic acid obtained by oxidation of methacrolein.

The alcohol to be reacted with methacrylic acid is not particularly limited, and examples thereof include methanol, ethanol, isopropanol, n-butanol, and isobutanol. Examples of the obtained methacrylic acid ester include methyl methacrylate, ethyl methacrylate, propyl methacrylate, and butyl methacrylate. The esterification reaction can be carried out in the presence of an acidic catalyst such as a sulfonic acid type cation exchange resin. The reaction temperature is preferably 50 to 200° C.

### EXAMPLES

Hereinafter, the present invention will be described in detail with reference to Examples and Comparative Examples, but the present invention is not limited to these Examples. In the Examples and Comparative Examples, “parts” means parts by mass.

### <Measurement of Particle Size Distribution of Slurry>

The particle size distribution of a slurry was measured by a laser diffraction type particle size distribution measurement method using a particle size distribution measurement device (trade name: SALD-7000) manufactured by Shimadzu Corporation. In the obtained volume-based particle size distribution, a peak width at half the height of the peak having the largest particle diameter was defined as a half-value width, and a particle diameter corresponding to a cumulative 50% by volume was defined as a median diameter.

### <Measurement of Viscosity>

The viscosity of a slurry was measured at 30° C. using a B-type viscometer (trade name: LVDV-II) manufactured by Brookfield Co., Ltd. The measurement was carried out at 30 rpm using Spindle No. 2.

### <Specific Gravity of Slurry>

The specific gravity of a slurry was calculated from the weight of the slurry having a volume of 100 ml filled in a 100 ml graduated cylinder.

### <Solid Content Concentration of Slurry>

The solid content concentration of a slurry was measured by using a moisture meter (trade name: MOC-120H) manufactured by Shimadzu Corporation and heating at 120° C. for 30 minutes.

### <Analysis of Raw Material Gas and Product>

Analysis of raw material gas and product was performed by gas chromatography (device: GC-2014 manufactured by Shimadzu Corporation, column: DB-FFAP manufactured by J&W, 30 m×0.32 mm, film thickness: 1.0 μm) From the analysis results of gas chromatography, a methacrolein conversion rate and a methacrylic acid selectivity were determined by the following formulas.

Methacrolein conversion rate (%)=((A−B)/A)×100

Methacrylic acid selectivity (%)=(D/C)×100

(In the formulas, A is the number of carbon atoms based on methacrolein in the raw material gas, B is the number of carbon atoms based on methacrolein in the reaction gas after the raw material gas passes through a catalyst and reacts, C is the number of carbon atoms based on the entire reaction product, and D is the number of carbon atoms based on methacrylic acid produced in the reaction gas after the raw material gas passes through a catalyst and reacts)

### Production Example 1

In the manufacturing apparatus shown in FIG. 1, 400 parts of pure water was charged into the tank 1, and further 100 parts of molybdenum trioxide, 7.0 parts of ammonium metavanadate, 8.0 parts of an 85% aqueous phosphoric acid solution, and 5.6 parts of copper (II) nitrate trihydrate were added. The mixture was heated to 95° C. while agitating with the agitator 11, and then agitated for 3 hours while maintaining the liquid temperature at 95° C. to obtain a slurry A1 containing a heteropolyacid.

The obtained slurry A1 had a half-value width α_{A1}=7.2 μm, a median diameter D_{A1}=3.2 μm, a viscosity of 15 cP, a specific gravity of 1.16 kg/L, and a solid content concentration of 20.2% by mass.

### Example 1

Using a volume V_{A1} of the slurry A1 obtained in Production Example 1, the slurry A1 was supplied to a turbo-type spiral pump 31, and was sent under the condition shown in Table 1. The discharge pressure of the pump 31 was measured by the pressure gauge 34. First, the slurry A1 was sent in a state where the valve 33 was switched to the line on the tank 1 side (pipe portion 32a) so that the slurry A1 circulates from the outlet 12 of the tank 1 to the liquid return port 13 at the upper part of the tank 1. Then, the valve 33 was switched to the line on the tank 2 side (pipe portion 32b), and the entire amount of the slurry A1 was fed from the liquid feed port 24 to the tank 2 to prepare a slurry A2. Table 1 shows the value of V_{POMP}/V_{A1} where the total volume of the slurry A1 supplied to the pump 31 is V_{POMP}.

Table 1 shows the particle size distribution of the obtained slurry A2 and the ratio α_{A2}/α_{A1} of the half-value widths of the particle size distributions of the slurry A1 and the slurry A2.

Next, a slurry C was prepared by mixing the slurry A2 and a raw material liquid containing a cationic raw material as follows. First, the slurry A2 in the tank 2 was held at 95° C., and 8.5 parts of cesium bicarbonate dissolved in 20 parts of pure water was added from the feed port 22 while agitating using the rotary blade agitator 21, and the mixture was agitated for 15 minutes. Thereafter, 15.0 parts of ammonium carbonate dissolved in 40 parts of pure water was added from the feed port 22 and agitated for 15 minutes to prepare the slurry C containing a heteropolyacid salt having a Keggin type structure.

Table 1 shows the pH, the half-value width and the median diameter of the obtained slurry C.

Then, the obtained slurry C was dried by a spray drying method to obtain a dried product. Subsequently, the obtained dried product was pressure-molded and then pulverized to obtain a molded article. The obtained molded article was filled in a cylindrical quartz glass calcination vessel having an inner diameter of 3 centimeters, heated under air flow at 10° C./h, and calcined at 380° C. for 15 hours to obtain a catalyst. The composition of the obtained catalyst excluding oxygen was Mo₁₂P_{1.2}V_{1.0}Cu_{0.4}Cs_{0.8}.

The obtained catalyst was filled in a reaction tube, and a raw material gas having 5% by volume of metachlorein, 10% by volume of oxygen, 30% by volume of water vapor, and 55% by volume of nitrogen was passed through at a reaction temperature of 285° C., and the reaction was carried out by adjusting the contact time between the raw material gas and the catalyst so that the metachlorein conversion was 40% and reacted.

The resulting product was collected and analyzed by gas chromatography to calculate methacrylic acid selectivity. The results obtained are shown in Table 1.

### Example 2

A catalyst for producing methacrylic acid was produced and the reaction was carried out using this catalyst to calculate the methacrylic acid selectivity by the same method as in Example 1 except that the slurry A1 obtained in Production Example 1 was sent to the pump 31 under the conditions shown in Table 1. The results obtained are shown in Table 1.

### Comparative Example 1

Without supplying the slurry A1 obtained in Production Example 1 to the pump 31, 8.5 parts of cesium bicarbonate dissolved in 20 parts of pure water was added to the slurry A1 and agitated for 15 minutes. Subsequently, 15.0 parts of ammonium carbonate dissolved in 40 parts of pure water was added and agitated for 15 minutes to obtain a slurry C. A catalyst was produced using the slurry C and the reaction was carried out using this catalyst to calculate the methacrylic acid selectivity by the same method as in Example 1. The results obtained are shown in Table 1.

### Example 3

A catalyst for producing methacrylic acid was produced and the reaction was carried out using this catalyst to calculate the methacrylic acid selectivity by the same method as in Example 1 except that the slurry A1 obtained in Production Example 1 was sent to the pump 31 under the conditions shown in Table 1. The results obtained are shown in Table 1.

### Example 4

A catalyst for producing methacrylic acid was produced and the reaction was carried out using this catalyst to calculate the methacrylic acid selectivity by the same method as in Example 1 except that the slurry A1 obtained in Production Example 1 was sent to the pump 31 under the conditions shown in Table 1. The results obtained are shown in Table 1.

### Comparative Example 2

Without supplying the slurry A1 obtained by Production Example 1 to the pump 31, the slurry A1 was agitated at a high speed by a homogenizer at 30° C. Thereafter, 8.5 parts of cesium bicarbonate dissolved in 20 parts of pure water was added and agitated for 15 minutes. Subsequently, addition of 15.0 parts of ammonium carbonate dissolved in 40 parts of pure water was added and agitated for 15 minutes to obtain a slurry C. A catalyst was produced using the slurry C and the reaction was carried out using this catalyst to calculate the methacrylic acid selectivity by the same method as in Example 1. The results obtained are shown in Table 1.

TABLE 1

| | Pump conditions | | | | Slurry A2 | | | | Slurry C | | | Methacrylic |
|---|---|---|---|---|---|---|---|---|---|---|---|---|
| | Feeding | Supply | Discharge | | Half-value | Median | | | | Half-value | Median | acid |
| | temperature | speed | pressure | V_{POMP}/ | width | diameter | | | | width | diameter | selectivity |
| | [° C.] | [L/min] | [kPa] | V_{A1} | α_{A2} [μm] | D_{A2} [μm] | α_{A2}/α_{A1} | D_{A2}/D_{A1} | pH | α_{C} [μm] | D_{C} [μm] | [%] |
| Example 1 | 95 | 222 | 500-520 | 3.8 | 5.6 | 3.0 | 0.78 | 0.94 | 2.5 | 5.5 | 3.0 | 94.1 |
| Example 2 | 95 | 164 | 500-520 | 3.1 | 6.3 | 3.0 | 0.88 | 0.94 | 2.6 | 7.1 | 3.7 | 94.6 |
| Example 3 | 95 | 229 | 500-520 | 1.0 | 5.6 | 3.0 | 0.78 | 0.94 | 2.5 | 6.6 | 3.0 | 94.1 |
| Example 4 | 95 | 15 | 500-520 | 7.0 | 5.2 | 2.7 | 0.72 | 0.84 | 2.7 | 6.9 | 2.6 | 93.5 |
| Comparative | — | — | — | — | 7.2 | 3.2 | 1.00 | 1.00 | 2.2 | 8.0 | 6.9 | 92.4 |
| Example 1 | | | | | | | | | | | | |
| Comparative | — | — | — | — | 5.0 | 1.7 | 0.69 | 0.53 | 2.3 | 12.6 | 2.1 | 91.1 |
| Example 2 | | | | | | | | | | | | |

As shown in Table 1, it was confirmed that a catalyst having a high methacrylic acid selectivity was obtained in Examples 1 to 4, in which the slurry A2 satisfying the predetermined particle size distribution was prepared. From these results, it can be seen that by preparing the slurry A2 satisfying the predetermined particle size distribution, a heteropolyacid salt can be uniformly formed in the preparation of the slurry C, and a desired catalyst for producing methacrylic acid can be produced.

In addition, as shown in Examples 1 to 4, when the slurry A2 is prepared by supplying the slurry A1 to the pump, a catalyst having a high methacrylic acid selectivity can be produced by adjusting V_{POMP}/V_{A} according to the supply speed of the slurry A1 to the pump.

On the other hand, in Comparative Example 1, in which the step of producing the slurry A2 having the predetermined particle size distribution was not performed, the methacrylic acid selectivity of the obtained catalyst became low.

In addition, as in Comparative Example 2, when a shearing force was applied to the slurry A1 using a homogenizer, the particles in the slurry A1 were broken, and as a result, it was not possible to prepare the slurry A2 satisfying the predetermined median diameter, and the methacrylic acid selectivity of the obtained catalyst became low.

### INDUSTRIAL APPLICABILITY

The present invention is industrially useful in that it is possible to provide a catalyst that enables a production of methacrylic acid with a high methacrylic acid selectivity.

### REFERENCE SIGNS LIST

1 tank

11 agitator

12 outlet

13 liquid return port

2 tank

21 agitator

22 feed port

23 outlet

24 liquid feed port

31 pump

32 pipe

32a, 32b pipe portion

33 two-way valve

34 pressure gauge

1. A method of producing a catalyst for producing methacrylic acid by oxidizing methacrolein, comprising:
preparing a slurry A1 containing a heteropolyacid containing at least phosphorus and molybdenum or a salt of the heteropolyacid containing at least phosphorus and molybdenum,
preparing a slurry A2 satisfying Formula (I) and Formula (II) using the slurry A1,
mixing the slurry A2 and a raw material liquid B containing a cationic raw material to prepare a slurry C, and
drying the slurry C,
α_{A2}/α_{A1}≤0.95 (I)
2 μm≤D_{A2}≤50 μm (II)
wherein, in Formula (I), am represents a half-value width of a particle size distribution of the slurry A1; and α_{A2} represents a half-value width of a particle size distribution of the slurry A2; and
in Formula (II), D_{A2} represents a median diameter of the particle size distribution of the slurry A2.

2. The method of producing a catalyst used for a production of methacrylic acid according to claim 1, wherein the the slurry C satisfies Formula (III):
2 μm≤D_{C}≤50 μm (III)
wherein, in Formula (III),
D_{C} represents a median diameter of a particle size distribution of the slurry C.

3. The method of producing a catalyst used-for a production of methacrylic acid according to claim 1, which satisfies Formula (IV):
0.6≤D_{A2}/D_{A1}<1.0 (IV)
wherein, in Formula (IV),
D_{A1} represents a median diameter (μm) of the particle size distribution of the slurry A1, and
D_{A2} represents the median diameter (μm) of the particle size distribution of the slurry A2.

4. The method of producing a catalyst used for a production of methacrylic acid according to claim 1, wherein the preparing the slurry A2 comprises supplying the slurry A1 to a pump.

5. The method of producing a catalyst used for a production of methacrylic acid according to claim 4, wherein, when a volume of the slurry A1 prepared in the preparing a slurry A1 is V_{A1}, and a total volume of the slurry A1 supplied to the pump is V_{POMP}, V_{POMP}/V_{A1} at beginning of the mixing the slurry A2 and the raw material liquid B is 0.1 or more.

6. The method of producing a catalyst used for a production of methacrylic acid according to claim 4, wherein V_{POMP}/V_{A1} at beginning of the mixing the slurry A2 and the raw material liquid B is 1.0 or more and 10.0 or less.

7. The method of producing a catalyst used for a production of methacrylic acid according to claim 4, wherein V_{POMP}/V_{A1} at beginning of mixing the slurry A2 and the raw material liquid B is larger than 1.0.

8. The method of producing a catalyst used for a production of methacrylic acid according to claim 4, wherein a supply speed of the slurry A1 to the pump is 1 L/min or more.

9. The method of producing a catalyst used for a production of methacrylic acid according to claim 4, wherein the pump is a turbo type pump or a reciprocating pump.

10. The method for producing a catalyst used for a production of methacrylic acid according to claim 4, wherein, when a tank for preparing the slurry A1 in the preparing a slurry A1 is tank 1, and a tank for mixing the slurry A2 and the raw material liquid B in the mixing the slurry A2 and the raw material liquid B is tank 2, the tank 1 and the tank 2 are different tanks.

11. The method of producing a catalyst used for a production of methacrylic acid according to claim 10, wherein the tank 1 and the tank 2 are connected through a pipe.

12. The method of producing a catalyst used for a production of methacrylic acid according to claim 11, wherein the pump is provided in the pipe.

13. The method of producing a catalyst used for a production of methacrylic acid according to claim 1, wherein the cation raw material comprises at least one selected from the group consisting of a compound containing an alkali metal and a compound containing an ammonium ion.

14. The method of producing a catalyst used for a production of methacrylic acid according to claim 1, wherein the slurry A1 has a viscosity of 1 to 200 cP at 30° C.

15. The method of producing a catalyst used for a production of methacrylic acid according to claim 1, wherein a solid content concentration of the slurry A1 is 5 to 60% by mass.

16. The method of producing a catalyst used for a production of methacrylic acid according to claim 1, wherein the catalyst comprises a compound represented by Formula (V):
PₐMo_{b}V_{c}Cu_{d}XₑY_{f}Z_{g}(NH₄)ₕOᵢ (V)
wherein, in Formula (V),
P, Mo, V, Cu, NH₄ and O represent phosphorus, molybdenum, vanadium, copper, ammonium and oxygen, respectively;
X represents at least one element selected from the group consisting of silicon, titanium, germanium, arsenic, antimony and bismuth;
Y represents at least one selected element selected from the group consisting of niobium, tantalum, tungsten, cerium, zirconium, silver, iron, zinc, chromium, magnesium, cobalt, manganese, barium and lanthanum;
Z represents at least one element selected from the group consisting of lithium, sodium, potassium, rubidium and cesium;
a to i represent molar ratios of each component; and
when b is 12 in Formula (V), a=0.5 to 3, c=0.01 to 3, d=0.01 to 2, e=0.1 to 3, f=0 to 3, g=0.01 to 3, h=0 to 20, and i represents a molar ratio of oxygen required to satisfy a valence of each of the components.

17. A method of producing methacrylic acid, comprising oxidizing methacrolein in a presence of the catalyst produced by the method according to claim 1.

18. A method of producing a methacrylic acid ester, comprising esterifying the methacrylic acid produced by the method according to claim 17.
